(19) United States
(12) Patent Application Publication
(10) Pub. No.: US 2004/0229793 A1
(43) Pub. Date: Nov. 18, 2004

(54) Compositions and methods for less immunogenic protein formulations

(76) Inventors: Sathyamangalam V. Balasubramanian, Amherst, NY (US); Robert M. Straubinger, Amherst, NY (US); Vivek S. Purohit, Fort Erie (CA); Karthik Ramani, Amherst, NY (US)

Correspondence Address: HODGSON RUSS LLP, ONE M & T PLAZA, SUITE 2000, BUFFALO, NY, 14203-2391, US

(21) Appl. No.: 10/773,075

(22) Filed: Feb. 5, 2004

Related U.S. Application Data
(60) Provisional application No. 60/445,134, filed on Feb. 5, 2003.

(51) Int. Cl. A61K 9/127; A61K 38/37

(52) U.S. Cl. 514/12; 424/450

(57) ABSTRACT

Composition and methods are described for low immunogenic protein formulations. An example of such a protein is antihemophilic factor (FVIII). The composition generally includes the protein, polypeptide or peptide, one or more agents that bind to epitope domains of the proteins to form a complex. Buffers containing salts may be used to stabilize this interaction. For example, Factor VIII and serine containing phospholipids in buffer salts containing Ca²⁺ and Na⁺ can be used to prepare protein-lipid structures. These complexes are useful for treatment of diseases such as Hemophilia. A method for the formation of novel non-liposomal structures is also disclosed.

This applications priority to U.S. provisional patent application No. 60/445,134 filed on Feb. 5, 2003, the disclosure of which is incorporated herein by reference.

This invention was made with Government support under grant no. R01HL-70227-01 from the National Institutes of Health. The Government has certain rights in the invention.

### FIELD OF THE INVENTION

The present invention relates to protein complexes having low immunogenicity and a method of making same.

### DISCUSSION OF RELATED ART

Hemophilia is a bleeding disorder caused by the deficiency of factor VIII (anti hemophilic factor, AHF or FVIII). FVIII is a multi domain protein comprising of six domains A1, A2, A3, B, C1 and C2 and activation of this protein by thrombin results in heavy (A1 and A2) and light chain (A3, C1 and C2) [1, 2]. Replacement therapy using blood concentrate, recombinant factor VIII and variants of factor VIII is the first line therapy for hemophilia. However, 15-35% of patients develop neutralizing antibodies and such immune response compromising therapy for hemophilia. Current treatment regimens to overcome the immune response exist but are not cost effective.

In general, it has been shown that the immune response to a therapeutic protein is due to the following reasons; (i) route of administration, (ii) existence of aggregates, (iii) frequent administration and (iv) specific epitope regions [3]. Recently, it has been shown that FVIII has a tendency to form aggregates [4] and the role of these aggregates in the development of immune response is not well understood. Further, there are primarily two epitope regions on FVIII molecule, C2 and A2 domains. Scandella et al [5] has shown that the antibody titre is highest against the C2 domain of the light chain. The C2 domain is also a membrane binding domain and it binds to phosphatidyl serine (PS) on platelet membranes as part of its coagulation cascade [6,7]. The anticoagulant action of antibodies to the C2 domain is due to inhibition of binding of factor VIII to phospholipid. It has been shown that the monoclonal antibodies against the C2 domain prevent the binding of Factor VIII to phospholipid containing membranes and based on these observations it was concluded that the epitope and membrane binding regions overlap [10, 11].

Previous studies on liposomal encapsulation of FVIII were aimed at increasing the encapsulation efficiency of FVIII (Factor VIII) in conventional liposomes and further to increase the in vivo stability and oral bioavailability [12, 13]. Due to the molecular architecture, lipid molecules tend to form several molecular assembles such as liposomes, micelles, non-bilayer structures and cochleate structures. Extensive studies have been done to use these molecular assemblies as drug delivery vehicles to improve the therapeutic properties of several drugs including proteins and peptides. These therapeutic properties are prolonging circulation time, reducing the toxicity, enhancement of immune response and reduction of in vivo degradation [14-25]. However, development of lipid complex to reduce immune response and antigenicity has not been investigated and therefore, there continues to be a need to develop approaches for reducing the immunogenicity of therapeutic proteins.

### SUMMARY OF THE INVENTION

The present invention discloses compositions having low antigenicity and immunogenicity and methods of making same. Accordingly, compositions comprising a therapeutic agent such as a protein, polypeptide or peptide and one or more molecules capable of binding to the protein (referred to herein as the binding agent) in such a way as to reduce its immunogenicity and antigenicity are disclosed. Such binding agents include phospholipids, particularly, serine containing phospholipids.

In one embodiment, the binding agent is a phospholipid. The protein-phospholipid complexes can be in the form of (1) liquid or freeze dried form of this liquid containing protein-binding agent complex (2) novel non-liposomal structures, (3) liposomes (4) micelles (5) cochleate (6) non-bilayer structures which reduce the immune response.

The present invention also discloses a method for reducing the immunogenicity and antigenicity of a protein by forming a complex with a serine containing phospholipid. The protein-phospholipid complex may be stabilized with suitable buffers. In one embodiment, the dried lipid film containing dimyristyl phosphatidyl choline (DMPC) and brain phosphatidyl serine (bPS) is hydrated using protein (such as FVIII) in various buffer systems. Novel, non liposomal structures are formed using DMPC, bPS in 300 mM NaCl and 5 mM CaCl₂. Conventional liposomes are formed as the buffer system is changed to water or phosphate buffered saline. This can also be accomplished by reducing the calcium or PS concentrations. The removal of DMPC and using 100% PS and a sonication or extrusion step leads to cochleate structures and use of PS with intermediate acyl chain length results in micellar structures. Use of shorter acyl chain length at lower concentrations yield protein-lipid complexes in solution. In all these compositions, it is considered that the protein-phospholipd complex shields the exposure of C2 domain.

### BRIEF DESCRIPTION OF THE DRAWINGS

FIG. 1 is the melting profile of FVIII at different heating rates.

FIG. 2 is the antibody binding assay that shows the conformational changes in the C2 domain.

FIG. 3 is a representation of size exclusion chromatography (SEC) profiles of Factor VIII in the presence or absence of O-Phospho-L-Serine.

FIG. 4 is a representation of antigenicity of FVIII-O,Phospho-L-Serine studied by sandwich ELISA.

FIG. 5 is a representation of folding studies of FVIII in O phospho L-Serine.

FIG. 6. is a representation of antigenicity of FVIII-PS complex in liposomes studied by sandwich ELISA. FVIII: Free FVIII, Invention FVIII: Composition used in the present invention and DMPC+FVIII: Physical Mixture of DMPC liposomes and FVIII lacking specific protein (FVIII) lipid (PS) complex.

FIG. 7A is a representation of the immune response in animal models for free FVIII and FVIII-PS complex. FVIII: Free FVIII, Invention FVIII: Composition used in the present invention.

FIG. 7B is a representation of FVIII and FVIII-PS complex liposomes in Factor VIII knockout Hemophilia A mice model.

FIG. 8 is the photograph of a Dextran density gradient showing the non-liposomal, low water volume fraction containing FVIII-PS complex.

### DETAILED DESCRIPTION OF THE INVENTION

The terms AHF, Factor VIII and FVIII are used interchangeably to mean the same molecule.

The present invention provides a method for reducing the antigenicity and immunogenicity of proteins. While the term “protein” is used throughout the application, it is intended to include peptides (generally considered to be 50 or less amino acids) as well as polypeptides (generally considered to be more than 50 amino acids).

The method of the present invention comprises the steps of forming complexes of one or more proteins, polypeptides or peptides with a phospholipid, preferably a phospholipid containing serine. Various types of protein-lipid structures can be formed depending upon the particular phospholipid, concentration and combinations of phospholipids

In general, the term “Liposome” means a generally spherical or spheroidal cluster or aggregate of lipid compounds, typically in the form of one or more concentric layers, for example, monolayers, bilayers or multi-layers. They may also be referred to as lipid vesicles. The liposomes may be formulated, for example, from ionic lipids and/or non-ionic lipids.

The terms “Cochleates” or “cochleate sturcutres” generally refer to a multilamellar lipid vesicle that is generally in the shape of a spiral or a tubule.

The term “Micelles” refers to colloidal entities formulated from lipids. Micelles may comprise a monolayer, bilayer, or hexagonal phase structure.

The use of phospholipids with no or short acyl chain length with 4 or less acyl chain carbon atoms will not lead to the formation of lipid molecular assemblies. The phospholipids containing intermediate acyl chain length between 5-12 acyl chian carbon atoms above its critical micellar concentration, form micelles and below micellar concentration form protein-lipid complexes. The phospholipids having longer acyl chain length (above 12 carbon atoms), due to the molecular architecture, tend to form several molecular assembles such as liposomes, non-bilayer structures and cochleate structures. The lipid composition may be varied to prepare liposome, non-bilayer structures and cochleate phases. A lipid composition of phosphatidyl choline (PC): phsophatidyl serine (PS) with high PC content will form liposomes upon hydration with buffers containing Ca²⁺ and Na+. The presence of Phosphatidyl Ethanolamine PE and PS promote the formation of non-bilayer structures. The formulation with PS (over 90 mol %) in the presence of Ca²⁺ with no or lower Na⁺ (100 MM) promote the formation of cochleate cylinders. Conditions such as temperature, Ca²⁺/Na²⁺ will be altered to reduce the size of cochleate cylinders in the nano-particles containing protein-lipid complex.

The phospholipids useful for the present invention are serine containing phospholipids. Examples of such serine containing phospholipids includes O-Phospho-L-Serine (OPLS), Dicaproyl Phosphatidyl Serine, Dioctanoyl Phosphotidyl Serine, Dimyristoyl, Dipalmitoyl, Dioleoyl-, Disteroyl- Phosphatidyl Serine. The serine containing phsopholipids may be used in combination with other molecules including other phospholipids. For example, phosphatidyl serine can be used in combination with phosphatidyl chloline or Phosphatidyl ethanolamine. The phosphotidyl serine may be obtained from any source such as natural (brain) or from synthetic origin. Phosphotidyl serine may be used in combination with phospatidyl choline (PC). The phosphotidyl choline may be dimyristoyl phophatidyl choline. The ratio of the PS and PC can be varied from 1:9 to 9:1. In one embodiment, the ratio is 3:7.

The protein-lipid compositions of the present invention are preferably stabilized and stored in suitable buffer systems. Such buffers include TRIS buffer and HEPES buffer and sodium and calcium salts. Optionally alcohol (such as 10% ethanol) may be added.

The protein-lipid complexes of the present invention can be characterized by standard methods. For example, fluorescence studies can be carried out on a SLM AMINCO 8000 series instrument or PTI 380 instrument using 4 nm as excitation and emission slits. The samples can be excited at 280 nm and the emission spectra scanned in the range of 300 to 400 nm. The emission spectra of free Factor VIII was observed around 335 nm and the addition of OPLS reduced the intensity of fluorescence emission indicating that the tertiary structure of the protein is not altered.

Further, the particle size of the lipid associated protein can be determined using a standard particle sizer (such as NICOMP 315 model). The particle size distribution can be analyzed using both Gaussian and NICOMP analysis for unimoidal and bimoidal distribution. The size of latex beads can be used as standard controls with each measurement.

The lipid structures can also be analyzed by negative staining electron microscopy. Such methods are routine in the art and can be used to confirm that there are no aggregates and to classify the structures as liposomes, non-bilayers or cochleates. The formation of non-bilayer and cochleate structures can also be investigated using Laurdan fluorescence. The lipid structures can be labeled with the probe by mixing the lipid containing solution with aqueous solution of the probe (containing 0.01% ethanol). The samples can be excited at 340 nm and the emission spectra were monitored at 440 nm. The excitation spectra can be acquired in the range of 320 and 420 nm, with emission monochromator at 440 nm.

The protein lipid complexes of the present invention can be delivered to an individual (such as an animal including a human being) by any standard means of administration such as intramuscular, intranasal, intraperitoneal, intravenous, oral, rectal, topical and the like. The complexes may be delivered directly to or near the target site or may be delivered directly or indirectly into the circulation. The complexes may be delivered in pharmaceutically acceptable carriers which are well known in the art.

The protein-lipid complexes of the present invention exhibit reduced immunogenicity as well as reduced antigenicity. Accordingly, such compositions can be used for reducing immune response in an individual against a therapeutic agent. The compositions of the present invention can also be used for delivery of a therapeutic agent to an individual in whom an immune reaction to the protein has already occurred. Thus, these composition can be used before or after the occurrence of an immune reaction.

In one embodiment, this invention provides specific FVIII-lipid complexes. The protein-lipid complexes may form novel, non-liposomal structures as well as structures such as liposomes, cochleate, micelles and non-bilayer structures to reduce the immune response and antigenicity. The method involves developing specific FVIII-lipid complex preferably stabilized by buffer conditions. Although not intending to be bound by any particular theory, it is believed that the reduction in antigenicity and immunogenecity arise from the protein lipid complex and not simply as a result of lipid molecular assemblies such as liposomes. These complexes have clotting activity in the presence of antibodies and exhibited reduced antigenicity as measured by their ability to bind to monoclonal antibodies in an ELISA assay. The complexes also showed reduced immune response in animal models. The present invention is useful not only to reduce the immune response development in previously untreated patients with FVIII but also retain FVIII clotting activity in previously treated patients who have developed antibodies.

Accordingly, in one embodiment, the present invention provides a method for reducing the immune response against FVIII. By the use of the compositions disclosed herein, immunogenicity is reduced while the clotting activity is maintained. It is considered that the reduction in immunigenicity is accomplished by complexing of the phospholipids with the C2 and A2 domains. It is considered that this decreases immunogenicity by (1) reducing aggregate formation, (2) decreasing the frequency of administration (the complexes alter the clearance mechanism thus by providing longer circulation time) and/or (3) shielding and altering the conformation of the epitope region. The improved pharmaceutical properties of the complex such as stability, altered clearance mechanism to increase circulation time and reduced antigenicity and immunogenicity is an unexpected observation.

The following examples are presented to further describe the invention and not intended to be restrictive in any way.

### EXAMPLE 1

This example demonstrates the preparation of protein-lipid complexes according to present invention. To illustrate this embodiment, FVIII was used. The FVIII-O-phospho-L-serine (OPLS) complex was formed by mixing 20 ug of the protein with 5 and 20 mM of the PS in 25 mM TRIS, 300 mM NaCl and 5 mM CaCl₂.

### EXAMPLE 2

This example describes the stability of the protein-lipid complexes of the present invention (Example. 1). For free FVIII the unfolding of the protein results in the aggregation of the protein and this in turn leads to the irreversibility of unfolding. The aggregation is initiated by small conformational changes in C2 domain. The unfolding/refolding studies were carried out with free FVIII and FVIII complexed to PS as described in Example 1 to determine the stability of the formulation containing protein and O-Phospho-L-Serine that is believed to bind to the C2 domain of FVIII.

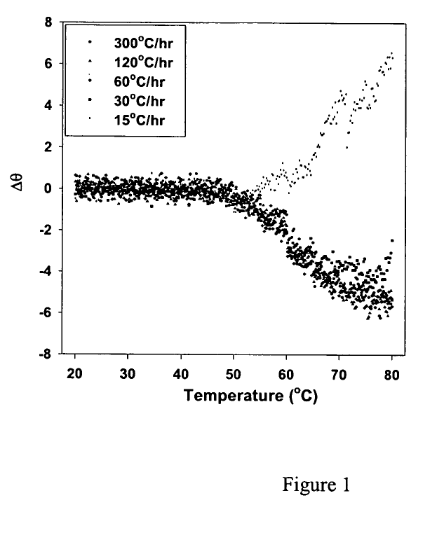

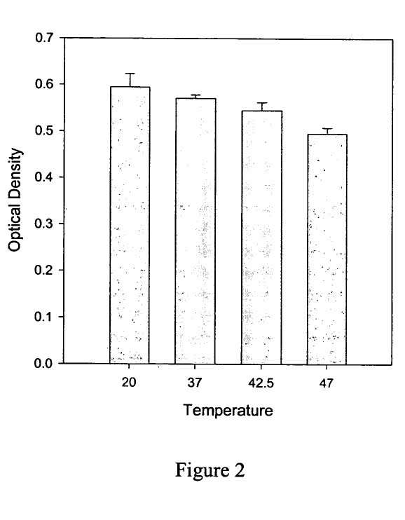

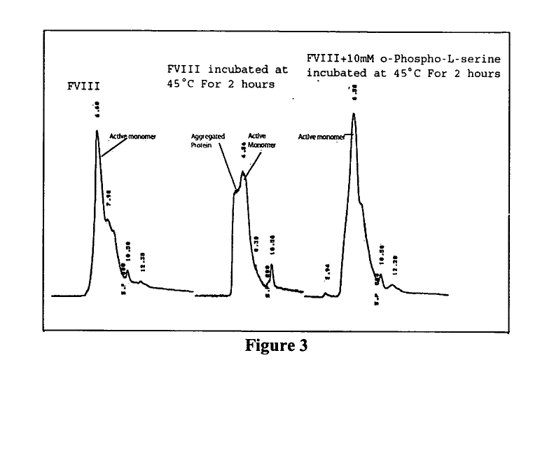

Circular Dichroism (CD), fluorescence anisotropy, size exclusion chromatography (SEC), domain specific antibody binding and clotting activity studies were carried out to investigate the temperature dependent physical and functional changes of recombinant human FVIII (rFVIII). Heating rate dependence of the unfolding profiles indicated that the thermal denaturation of the protein was at least in part under kinetic control (FIG. 1). A folding model was proposed to explain the aggregation kinetics of Factor VIII. Based on this model, unfolding of Factor VIII was interpreted in terms of the simple two-state kinetic model, Aggregated (A) → Native (N) where k is a first-order kinetic constant that changes with temperature, as given by the Arrhenius equation. The activation energy associated with the above transition was calculated to be ˜127.98 Kcal/Mole (˜534.97 KJ/Mole). Antibody binding studies indicated that conformational changes in the lipid-binding region (2303-2332) of the C2 domain may at least in part be responsible for the initiation of aggregation (FIG. 2).. Analysis of the SEC profile of FVIII in the presence and in the absence of OPLS clearly showed that the monomeric population is significantly higher than that of aggregated protein in the presence of PS, possibly due to the interference of OPLS in the aggregation kinetics of Factor VIII (FIG. 3). The data indicates that the complex improves the stability of FVIII and may help to reduce the immunogenecity by reducing the aggregates

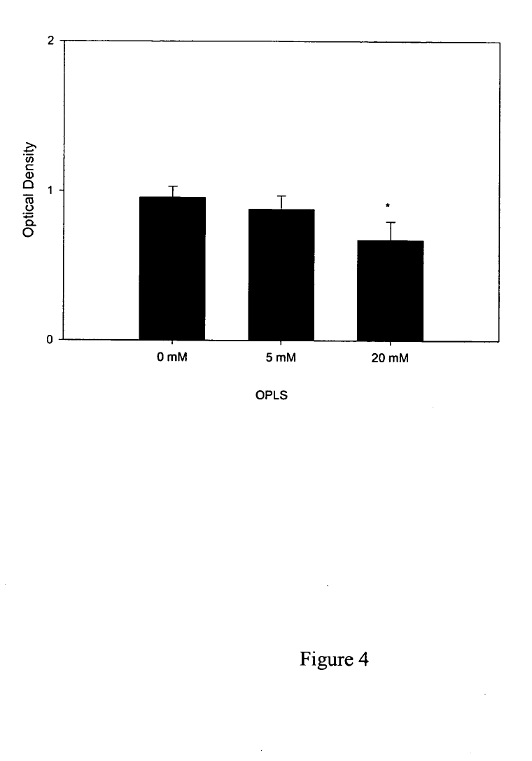

For determining the folding and unfolding, CD spectral studies were carried out. CD spectra were acquired on a JASCO-715 spectropolarimeter calibrated with d10 camphor sulfonic acid. Samples were scanned in the range of 255 to 208 nm for secondary structure analysis, and typically, the protein concentration used was 20-22 μg/ml. CD spectra of the protein were corrected by subtracting the spectrum of the buffer baseline. Multiple scans were acquired and averaged to improve signal quality. Melting of the protein was followed over the temperature range of 20° C.-80° C. with a 2 min holding time at every 2.5° C. The temperature scans were acquired with a Peltier 300 RTS unit and the profiles were generated using the software provided by the manufacturer. The results are shown in FIG. 4. The recovery of native like structures were not possible for excipient free protein due to aggregation whereas presence of O-Phospho-L-Serine resulted in substantial recovery of native structure.

O-Phospho-L-Serine, PS containing conventional liposomes and cochleate particles were also investigated as complexing agents to improve the stability.

### EXAMPLE 3

This example demonstrates that the protein-lipid composition of the present invention (Example. 1) reduces the immunogenicity against the protein in Sprague-Dawley rats. To illustrate this embodiment, OPLS-Factor VIII complex was administered to Sprague-Dawley rats. This rat model has been shown to be suitable to study antibody development to FVIII. The antibody titer measured by ELISA for free FVIII and FVIII-PS complex. Two weeks after the administration, the analysis of antibody titer for Factor VIII -OPLS complex was found to be non-immunotoxic. The antibody titers for free FVIII is 563.72±916.15 and no detectable antibody titers was observed for FVIII-OPLS complex.

### EXAMPLE 4

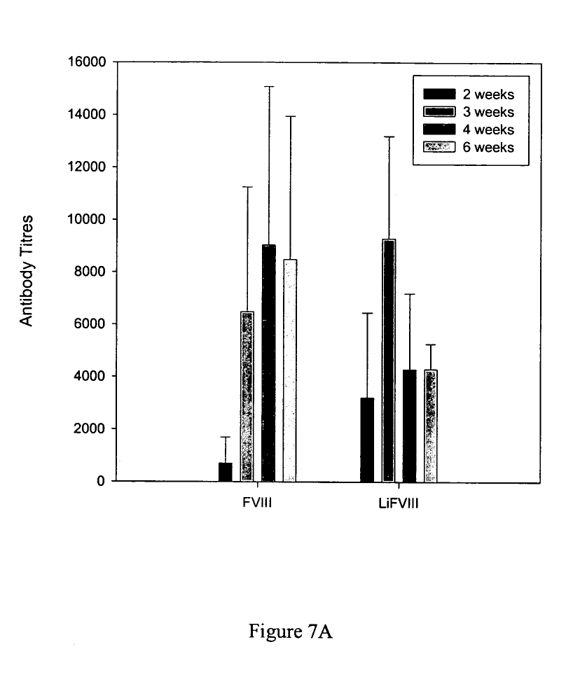

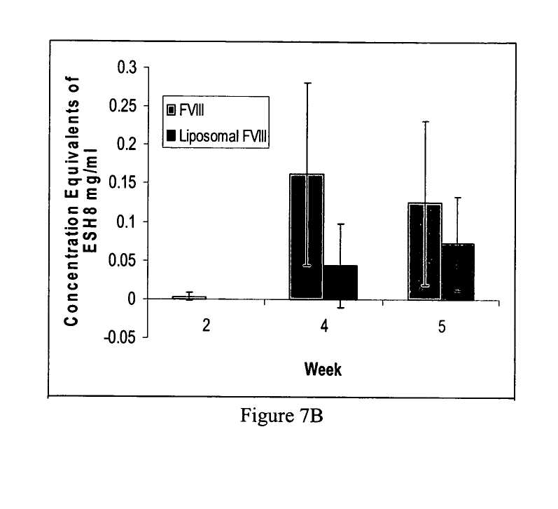

This example demonstrates that the composition of the present invention can be made as small unilamellar vesicles. 0.3 mg/ml of DMPC and 0.15 mg/ml of bPS dissolved in a round bottomed flask and the solvent was evaporated to form a thin film. The film was then hydrated to form MLV's and the MLVs were extruded through 200 nm polycarbonate filters to form SUV's in the size range of 160 nm. The immune response of this formulation is described in FIG. 7 and 7b.

### EXAMPLE 5

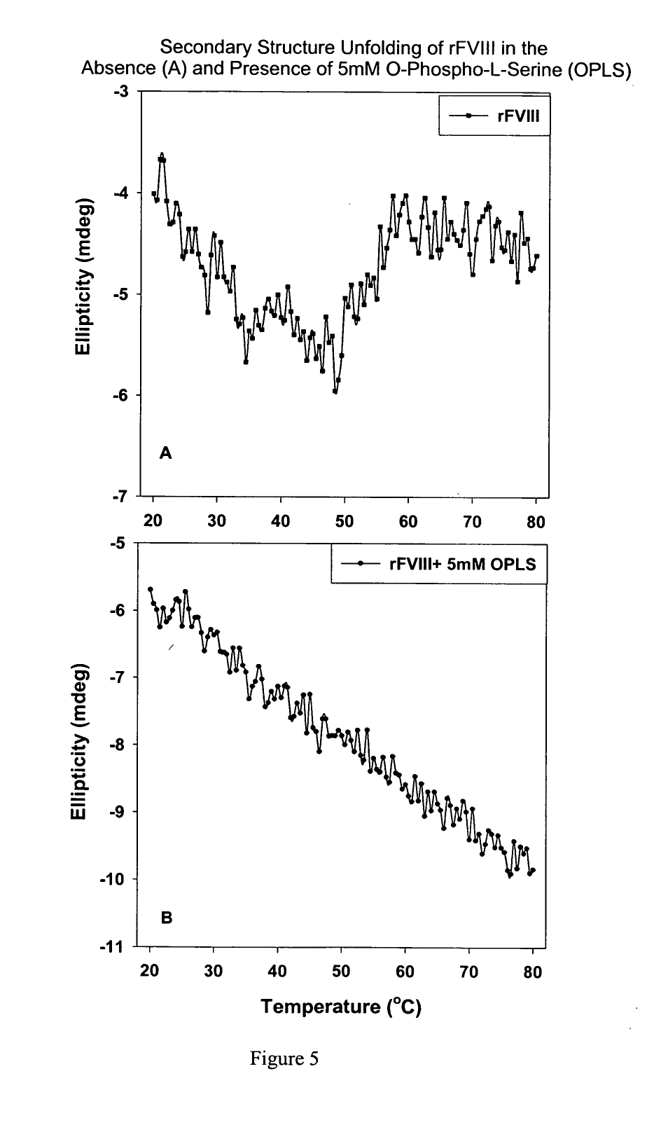

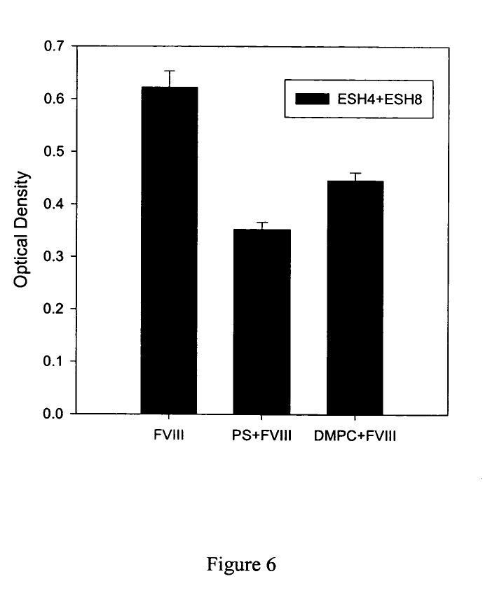

This example demonstrates the general methodology of the ELISA assay to investigate the antigenicity and the participation of particular epitope region of the protein in forming the complex. The antibody binding of the protein-lipid complexes was investigated by antibody capture ELISA and sandwich ELISA. For Sandwich ELISA, 96 well plates (Nunc-Maxisorb) were coated with an anti-C2 domain antibody (ESH4) by incubating 50 μl/well solution of the antibody at a concentration of 5 μg/ml in carbonate buffer (0.2 M, pH 9.4) overnight at 4° C. The plate was then washed 10 times with 100 μl of Phosphate buffer containing 0.05% Tween 20 (PBT consisting of 10 mM Na₂HPO₄, 1.8 mM KH₂PO₄, 0.14 mM NaCl, 2.7 mM KCl, and 0.02% NaN₃). The remaining nonspecific protein binding sites on the plastic's adsorptive surface were blocked by incubating 200 μl of blocking buffer consisting of 1% bovine serum albumin in phosphate buffer (PB consisting of 10 mM Na₂HPO₄, 1.8 mM KH₂PO₄, 0.14 mM NaCl, and 2.7 mM KCl) for 2 hours at room temperature. The plates were washed 10 times with PBT and 50 μl of 100 ng/ml of rFVIII or OPLS/Liposome associated rFVIII (Examples 1 and 3) in blocking buffer was added and incubated at 37° C. for 1 hour. The plates were washed 10 times with PBT and incubated with 50 μl of biotinylated ESH8—another anti-C2 antibody, at 1 μg/ml concentration and 50 μl of a 1:1000 dilution of avidin-alkaline phosphatase conjugate, both in blocking buffer at room temperature for 1 hour. The plates were washed 10 times with PBT and 100 μl of 1 mg/ml p-nitrophenyl phosphate solution in diethanolamine buffer (consisting of 1 M diethanolamine, 0.5 mM MgCl₂ and 0.02% NaN₃). The plates were incubated at room temperature for 30 minutes and the reaction was quenched by adding 100 μl of 3 N NaOH. Absorbance was read by a plate reader at 405 nm. The ELISA studies indicated that less C2 domain specific antibodies, ESH8 or ESH4 bound to the protein in the presence of PS (OPLS or liposomes FIG. 5). The results showed that the binding of these monoclonal antibodies were inhibited by lipid suggesting that epitope regions are shielded in the protein-lipid complex. Further, the specific complex FVIII-PS is responsible for the the epitope shielding is further confirmed by the control experiment in which the antigenicity was observed only with DMPC/PS mixture but not with DMPC alone (FIG. 6).

### EXAMPLE. 6

This example demonstrates that the protein-lipid composition of the present invention (Example. 4) reduces the immunogenicity against the protein in animal models, Sprague-Dawley rats and Factor VIII knock out mice, Hemophilia A model.

The antibody titres evaluated in Sprague-Dawley rats at 4^{th} and 6^{th} weeks post administration of the protein-liposomes complex was found to be lower for FVIII-PS complex compared to free FVIII (FIG. 7A).

In another illustration of this embodiment, the effect of protein-lipid complexes of the present invention on immunogenicity was tested in a Factor VIII knock out mice model. The phenotype of the mice is severe hemophilia. (exon 16 knock-out by targeted disruption using a neo cassette). The protein-liposome complex was administered sc and the antibody titres were measured using ELISA assay. As is clear from the FIG. 7B, the antibody concentrations were lower for the liposome bound protein after 5 weeks of administration. The data clearly demonstrates that liposome bound Factor VIII displays less inhibitors compared to free factor VIII. This observations may partly be due to altered (1) conformation and aggregation kinetics of free FVIII, (2) processing by immune system and (3) clearance mechanism.

In all the above mentioned studies, control experiments were performed using PS free conditions and the concentration of the protein associated with the lipid and lipidic structures are extremely low indicating that the presence of specific lipid-FVIII complex in all these lipidic formulations. Further, the monoclonal antibody specific for C2 domain did not bind to FVIII in the presence of Serine clearly indicates the presence of complex in lipid structures studied.

### EXAMPLE 7

The following examples illustrate the formation of protein-lipid complexes in micelles, non-bilayered structures, cochleate structures and in novel non-liposomal lipid particles. This example describes the formation of micelles by the compositions described herein. The protein solution was mixed with shorter acyl chain lipids such as Dihexanoyl phosphatidyl Serine (below and above 0.3 mM) at lower and higher concentration (below and above critical micellar concentrations) and the resulting micellar particles were characterized. The structure of the micelles were characterized by light scattering, circular dichroism and fluorescence studies. The functional assays such as activity and antibody binding were carried out.

### EXAMPLE 8

This example describes the formation of cochleate structures by the compositions described herein. 0.15 mg/ml of bPS was dissolved in chloroform and the solvent was evaporated to form a thin lipid film. The film was then hydrated in several buffer system at pH 7.0 and the MLVs were either extruded or sonicated to form SUVs. The resulting SUVs were mixed with protein in buffer system containing 5 mM CaCl₂ to form cochleate structure. The lipid structures were analyzed by light scattering, differential interference microscopy, negative stain electron microscopy and by fluorescence studies. These studies showed that the lipid structures formed by this procedure were cochleate in nature.

### EXAMPLE 9

This example is another illustration of the preparation of protein-lipid complexes of the present invention. 0.15 mg/ml of bPS and 0.3 mg.ml of di oleoyl phosphatidyl ethanol amine (DOPE) was dissolved in chloroform and the solvent was evaporated to form a thin lipid film. The resulting lipid film was hydrated with phosphate buffered saline to form hexagonal phases. The non-bilayer structures were characterized by fluorescence studies.

### EXAMPLE 10

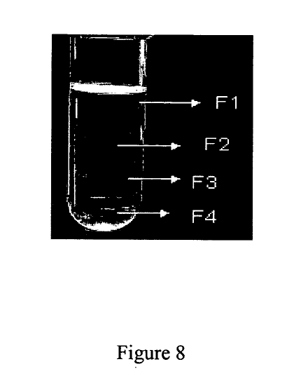

This example is another illustration of the preparation of protein-lipid complexes of the present invention in a novel non-liposomal structures. 0.3 mg/ml of DMPC, 0.15 mg/ml of bPS were dissolved in chloroform and the solvent was evaporated to form a thin lipid film in a round bottom flask. The lipid was hydrated using a buffer system containing FVIII, 25 mM TRIS, 300 mM NaCl and 5 mM CaCl₂ and the solution was gently swirled either at room temperature at 37° C. The film was then hydrated in appropriate buffer (25 mM TRIS, 300 mM NaCl and 5 mM CaCl₂), with gentle swirling. The MLV's thus formed were subjected to dextran centrifugation gradient to separate the free protein from protein associated with MLV's. 0.5 ml of the lipid associated protein was mixed with 1 ml of 20% w/v of dextran and a 3 ml of 10% w/v dextran was layered over the above solution. Then 0.5 ml of buffer layered on top. The gradient was centrifuged for 35 min at 45K RPM using Beckman SW50.1 rotor. The results of the centrifugation study is shown in FIG. 8. As is clear from the figure, there are some lipidic fractions that could not be floated and are denoted as fraction 3 in the figure. This fraction was observed at the interface of 14% and 10% dextran. The fact that this fraction could not be floated indicates that this lipidic fraction does not have enough buyoancy or encapsulated water. Conventional liposomes generally float to the top of the gradient because of their entrapped water. Therefore, the fraction that does not float may be a non-liposomal protein containing lipidic particles. The fraction was collected and tested for lipid content by mass spectrometry and for protein content by activity. The mass spectroscopy studies showed that this fraction contained lipids including high PC content suggesting that it is not just PS-Ca+ complex. The activity assay showed approximately 40% of the initial protein was encapsulated in this fraction.

There are several possible explanations for the dense fraction 3 which has no or little water content. This fraction may represent: (1) very small unilamellar vesicles with less encapsulated water volume. In order to determine if smaller vesicles (less than 200 nm) could be floated under identical conditions, pre sized liposomes were prepared by extruding through polycarbonate filters. The extrusion was repeated 3 times and the size of the particles was determined to be around 160 nm. The resulting SUVs were mixed with FVIII and were subjected to Dextran centrifugation gradient. This control study showed that these SUV's did not show fraction 3 band indicating that the observation of such bands is not due to the formation of small liposomes. Further, this experiment was performed under identical buffer and experimental conditions to rule out any artifacts in the dextran gradient. (2) The second possibility is that fraction 3 may represent the formation of cochleate structures, which have less water content [26]. However, the formation of cochleate structures needs a very high PS content (>50%). In the formation of fraction 3 band, the PS content used was around 30% and under these conditions the formation of cochleate structures has not been shown. (3) The formation of collapsed Ca(PS)2 complex that has a dehydrated structure [27] may not float and can form a dense band. The formation of such collapsed structure requires very high PS content (>50%) but in the absence of Na+. However, in the present example, the composition contains low PS content and a very high concentration of Na+ i.e., 30% PS and 300 mM NaCl is used and therefore, the possibility of Ca(PS)2 formation is ruled out. (4) The PS and calcium system has been shown to promote vesicle fusion. However, the fusion of vesicles by divalent cations such as Ca²⁺ is inhibited by the presence of Na+ as it competes with calcium for the lipid binding site. The estimated amount of Calcium bound per PS in this PC/PS ratio of 7:3 and in the presence of 300 mM NaCl and 5 mM CaCl₂, is between 0.22 (500 mM NaCl) to 0.35 (100 mM NaCl) [28, 29]. This estimated bound calcium per PS is less than the critical ratio of 0.35 to 0.39 required for fusion in a small unilamellar PS/PC vesicle system. Thus, the fusion of vesicles does not appear to be represent the dense band. This is because the larger PC fraction (>50%) may result in less PS-divalent cation complex and its ability to cluster into large domains to induce fusion)[28, 29]. Based on these arguments, it appears that the dense band may be due to the formation of novel, non-liposomal lipid particles. This band is non-liposomal because of less encapsulated water volume. In order to understand the structure of this novel lipidic structure, negative stain electron microscopy, differential contrast interference optical microscopy, light scattering, circular dichroism and fluorescence measurements (data not shown) were performed.

Centrifugation studies carried out under several conditions indicate that buffer conditions, bulk protein concentration, use of alochol (such as 10% ethanol) and protein-lipid ratio can be varied to obtain the desired yield of the complexes. Such variations in these parameters are considered to be within the purview of one skilled in the art. In general, it was observed that higher protein concentration resulted in more intense dense fraction as confirmed by visual inspection.

### EXAMPLE 11

This example demonstrates that the protein-lipid compositions of the present invention retain their biological activity. To illustrate this embodiment, the effect of protein-lipid complex comprising Factor VIII on clotting was tested. rFVIII clotting activity was determined by one-stage activated partial thromboplastin time (APTT) assay using micronized silica as activator and FVIII deficient plasma as the substrate. The APTT assay was performed using a COAG-A-MATE model coagulation analyzer (Organon Teknika Corporation, Durham, N.C.). Briefly, rFVIII was added to FVIII deficient plasma and the clotting time was monitored. The activity of the rFVIII was then obtained from calibration curve constructed using the clotting times determined from various dilutions of a lyophilized reference concentrate of known activity. The concentration of the protein was determined independently using Bicinchoninic acid (BCA) assay and compared with activity. For example, all the 20-22 μg/ml of the protein corresponds to specific activity of 87-95.6 IU. The stock solution used to prepare the samples had a specific activity of 2174 IU/0.5 mg/ml.

While this invention has been described through examples presented herein, routine modifications can be made to the invention without departing from the spirit of the invention. Such modifications are intended to be within the scope of the claims.

References

1. Fay, P. J. Factor VIII structure and function. Thromb Haemost 70, 63-7 (1993).

2. Foster, P. A. & Zimmerman, T. S. Factor VIII structure and function. Blood Rev 3, 180-91 (1989).

3. Braun, A., Kwee, L., Labow, M. A. & Alsenz, J. Protein aggregates seem to play a key role among the parameters influencing the antigenicity of interferon alpha (IFN-alpha) in normal and transgenic mice. Pharmaceutical Research 14, 1472-8 (1997).

4. Grillo, A. O. et al. Conformational origin of the aggregation of recombinant human factor VIII. Biochemistry 40, 586-95. (2001).

5. Scandella, D. H. et al. In hemophilia a and autoantibody inhibitor patients: the factor viii a2 domain and light chain are most immunogenic. Thromb Res 101, 377-85. (2001).

6. Lenting, P. J. et al. The light chain of factor VIII comprises a binding site for low density lipoprotein receptor-related protein. Journal of Biological Chemistry. 274, 23734-9 (1999).

7. Saenko, E. L., Yakhyaev, A. V., Mikhailenko, I., Strickland, D. K. & Sarafanov, A. G. Role of the low density lipoprotein-related protein receptor in mediation of factor VIII catabolism. Journal of Biological Chemistry. 274, 37685-92 (1999).

8. Gilbert, G. E., Furie, B. C. & Furie, B. Binding of human factor VIII to phospholipid vesicles. J Biol Chem 265, 815-22 (1990).

9. Stoilova-McPhie, S., Villoutreix, B. O., Mertens, K., Kemball-Cook, G. & Holzenburg, A. 3-Dimensional structure of membrane-bound coagulation factor VIII: modeling of the factor VIII heterodimer within a 3-dimensional density map derived by electron crystallography. Blood 99, 1215-23. (2002).

10. Scandella, D. et al. Some factor VIII inhibitor antibodies recognize a common epitope corresponding to C2 domain amino acids 2248 through 2312, which overlap a phospholipid-binding site. Blood 86, 1811-9. (1995).

11. Barrow, R. T., Healey, J. F., Jacquemin, M. G., Saint-Remy, J. M. & Lollar, P. Antigenicity of putative phospholipid membrane-binding residues in factor VIII. Blood 97, 169-74. (2001).

12. Huong, T. M., Ishida, T., Harashima, H. & Kiwada, H. The complement system enhances the clearance of phosphatidylserine (PS)-liposomes in rat and guinea pig. Int J Pharm 215, 197-205. (2001).

13. Dal Monte, P. & Szoka, F. C., Jr. Effect of liposome encapsulation on antigen presentation in vitro. Comparison of presentation by peritoneal macrophages and B cell tumors. J Immunol 142, 1437-43. (1989).

14. Heath, T. D., Edwards, D. C. & Ryman, B. E. The adjuvant properties of liposomes. Biochemical Society Transactions 4, 129-33 (1976).

15. Kirby, C. J. & Gregoriadis, G. Preparation of liposomes containing factor VIII for oral treatment of haemophilia. J Microencapsul 1, 33-45. (1984).

16. Hemker, H. C., Hermens, W. T., Muller, A. D. & Zwaal, R. F. Oral treatment of haemophilia A by gastrointestinal absorption of factor VIII entrapped in liposomes. Lancet 1, 70-1. (1980).

17. Martin, F. J., Hubbell, W. L. & Papahadjopoulos, D. Immunospecific targeting of liposomes to cells: a novel and efficient method for covalent attachment of Fab′ fragments via disulfide bonds. Biochemistry 20, 4229-38. (1981).

18. Matthay, K. K., Heath, T. D., Badger, C. C., Bernstein, I. D. & Papahadjopoulos, D. Antibody-directed liposomes: comparison of various ligands for association, endocytosis, and drug delivery. Cancer Res 46, 4904-10. (1986).

19. Olson, F., Hunt, C. A., Szoka, F. C., Vail, W. J. & Papahadjopoulos, D. Preparation of liposomes of defined size distribution by extrusion through polycarbonate membranes. Biochim Biophys Acta 557, 9-23. (1979).

20. Papahadjopoulos, D. et al. Sterically stabilized liposomes: improvements in pharmacokinetics and antitumor therapeutic efficacy. Proc Natl Acad Sci U S A 88, 11460-4 (1991).

21. Phillips, N. C. & Emili, A. Enhanced antibody response to liposome-associated protein antigens: preferential stimulation of IgG2a/b production. Vaccine 10, 151-8. (1992).

22. Storm, G., Wilms, H. P. & Crommelin, D. J. Liposomes and biotherapeutics. Biotherapy 3, 25-42 (1991).

23. Szoka, F., Jr. & Papahadjopoulos, D. Procedure for preparation of liposomes with large internal aqueous space and high capture by reverse-phase evaporation. Proc Natl Acad Sci U S A 75, 4194-8. (1978).

24. Szoka, F. et al. Preparation of unilamellar liposomes of intermediate size (0.1-0.2 mumol) by a combination of reverse phase evaporation and extrusion through polycarbonate membranes. Biochim Biophys Acta 601, 559-71. (1980).

25. Van Slooten, M. L. et al. Liposomes as sustained release system for human interferon-gamma: biopharmaceutical aspects. Biochim Biophys Acta 1530, 134-45. (2001).

26. Papahadjopoulos, D., Vail, W. J., Jacobson, K. & Poste, G. Cochleate lipid cylinders: formation by fusion of unilamellar lipid vesicles. Biochim Biophys Acta 394, 483-91. (1975).

27. Coorssen, J. R. & Rand, R. P. Structural effects of neutral lipids on divalent cation-induced interactions of phosphatidylserine-containing bilayers. Biophys J 68, 1009-18. (1995).

28. Duzgunes, N. et al. Calcium- and magnesium-induced fusion of mixed phosphatidylserine/phosphatidylcholine vesicles: effect of ion binding. J Membr Biol 59, 115-25. (1981).

29. Duzgunes, N., Wilschut, J., Fraley, R. & Papahadjopoulos, D. Studies on the mechanism of membrane fusion. Role of head-group composition in calcium- and magnesium-induced fusion of mixed phospholipid vesicles. Biochim Biophys Acta 642, 182-95. (1981).

1. A method for reducing the antigenicity of a therapeutic agent selected from the group consisting of peptide, polypeptide and protein comprising the steps of forming a complex of the therapeutic agent with a serine containing phospholipid in the presence of sodium and calcium salts, wherein the antigenicty of the therapeutic agent is reduced when complexed with the serine containing phospholipid.

2. The method of claim 1, wherein the complex improves the stability of the therapeutic agent.

3. The method of claim 1, wherein the complex runs at the interface of 10% and 14% dextran gradient.

4. The method of claim 1, wherein the complex is selected from the group consisting of liposomes, micelles, cochleates and non-bilayer structures.

5. The method of claim 1, wherein the phospholipid is phosphatidyl serine.

6. The method of claim 5, wherein the phospholipid further comprises phosphatidyl choline or phosphatidyl ethanolamine.

7. The method of claim 6, wherein the ratio of phosphatidyl choline to phospatidyl serine is 7:3.

8. The method of claim 1, wherein the sodium salt is about 100 mM NaCl and the calcium salt is about 5 mM CaCl₂.

9. The method of claim 1, wherein the therapeutic agent is Factor VIII.

10. A method for reducing the immunogenicity of a therapeutic agent selected from the group consisting of peptide, polypeptide and protein comprising the steps of forming a complex of the therapeutic agent with a serine containing phospholipid in the presence of sodium and calcium salts, wherein the immunogenicity of the therapeutic agent upon administration to an individual is reduced when complexed with the serine containing phospholipid.

11. The method of claim 10, wherein the complex runs at the interface of 10% and 14% dextran gradient.

12. The method of claim 10, wherein the complex is selected from the group consisting of liposomes, micelles, cochleates and non-bilayer structures.

13. The method of claim 10, wherein the phospholipid is phosphatidyl serine.

14. The method of claim 13, wherein the phospholipid further comprises phosphatidyl choline or phosphatidyl ethanolamine.

15. The method of claim 14, wherein the ratio of phosphatidyl choline to phospatidyl serine is 7:3.

16. The method of claim 10, wherein the sodium salt is about 100 mM NaCl and the calcium salt is about 5 mM CaCl₂.

17. The method of claim 10, wherein the therapeutic agent is Factor VIII.

18. Lipid-protein complexes which are formed by incubating a protein with a serine containing phospholipid in the presence of about 100 nM of a sodium salt and about 5 nM of a calcium salt and characterized in that the complexes migrate at the interface of 10% and 14% dextran gradient.

19. The lipid-protein complexes of claim 18 wherein the serine containing phospholipid is phosphatidyl serine.

20. The lipid-protein complexes of claim 19 further comprising phosphatidyl choline and wherein the ratio of phosphatidyl choline to phosphatidyl serine is 7:1.

21. The lipid-protein complex of claim 20 wherein the protein is Factor VIII.
